(19) United States
(12) Patent Application Publication
(10) Pub. No.: US 2024/0399137 A1
(43) Pub. Date: Dec. 5, 2024

(54) DELIVERY SYSTEM WITH EXPANDABLE DISTAL CAPSULE

(71) Applicant: EDWARDS LIFESCIENCES CORPORATION, Irvine, CA (US)

(72) Inventors: Michael G. Valdez, Riverside, CA (US); Hien T. Nguyen, Aliso Viejo, CA (US)

(21) Appl. No.: 18/798,546

(22) Filed: Aug. 8, 2024

Related U.S. Application Data
(63) Continuation of application No. PCT/US2023/012573, filed on Feb. 8, 2023.
(60) Provisional application No. 63/309,290, filed on Feb. 11, 2022.

(51) Int. Cl. A61M 60/865; A61M 60/139

(52) U.S. Cl. CPC A61M 60/865; A61M 60/139; A61M 2210/127

(57) ABSTRACT

An implant delivery system comprises a distal portion configured to naturally assume a first width and expand beyond the first width in response to an implant device being inserted into the distal portion via an opening at a distal end of the distal portion and a proximal portion configured to generally maintain the first width

### RELATED APPLICATION

This application is a continuation of International Patent Application No. PCT/US2023/012573, filed Feb. 8, 2023 and entitled DELIVERY SYSTEM WITH EXPANDABLE DISTAL CAPSULE, which claims priority to U.S. Provisional Application No. 63/309,290, filed Feb. 11, 2022, and entitled DELIVERY SYSTEM WITH EXPANDABLE DISTAL CAPSULE, the disclosure of which is hereby incorporated by reference in its entirety.

### BACKGROUND

### Field

The present disclosure generally relates to the field of a medical implant delivery devices.

### Description of Related Art

Various medical procedures involve the implantation of medical implant devices within the anatomy of the heart. Medical implant devices can be delivered into a patient's body and/or heart for various purposes, which can include supporting blood circulation.

### SUMMARY

Described herein are one or more methods and/or devices to facilitate delivery of various medical implant devices, which can include mechanical circulatory systems.

Some implementations of the present disclosure relate to an implant delivery system comprising a distal portion configured to naturally assume a first width and expand beyond the first width in response to an implant device being inserted into the distal portion via an opening at a distal end of the distal portion and a proximal portion configured to generally maintain the first width.

The proximal portion may not be configured to expand in response to the implant device extending into or against the proximal portion. In some examples, the distal portion comprises one or more bendable rods configured to bend in response to the implant device being inserted into the distal portion.

In some examples, the distal portion further comprises a first coating configured to extend across the one or more bendable rods to form an inner diameter of the distal portion. The distal portion may further comprise a second coating configured to extend across the one or more bendable rods to form an outer diameter of the distal portion.

The one or more bendable rods may be evenly spaced around a circumference of the distal portion. In some examples, the one or more bendable rods extend longitudinally along the distal portion.

In some examples, the proximal portion comprises a braided network of fibers. The implant delivery system may further comprise a nose cone configured to provide a leading end for the implant device.

The nose cone may be configured to extend at least partially over the distal end of the distal portion. In some examples, the nose cone is configured to extend from the implant device.

In some examples, the proximal portion is configured to receive a tubular portion of the implant device. The tubular portion may have a generally flexible structure. In some examples, the tubular portion is configured to bend to facilitate navigation of the implant device through an aortic arch.

In accordance with some implementations of the present disclosure, a method comprises delivering a delivery device carrying an implant device into a blood vessel. The delivery device comprises a distal portion configured to naturally assume a first width and expand beyond the first width in response to a distal member of the implant device being inserted into the distal portion via an opening at a distal end of the distal portion and a proximal portion configured to generally maintain the first width. The method further comprises extending at least the distal member of the implant device beyond the distal end of the delivery device to cause the distal portion of the delivery device to assume the first width and retracting at least the distal member of the implant device into the distal portion of the delivery device to cause the distal portion of the delivery device to expand beyond the first width.

In some examples, the proximal portion is not configured to expand in response to the distal member of the implant device retracting into the distal portion of the delivery device. The distal portion may comprise one or more bendable rods configured to bend in response to the implant device being inserted into the distal portion.

The distal portion may comprise a first coating configured to extend across the one or more bendable rods to form an inner diameter of the distal portion. In some examples, the distal portion further comprises a second coating configured to extend across the one or more bendable rods to form an outer diameter of the distal portion.

In some examples, the proximal portion comprises a braided network of fibers. The proximal portion may be configured to receive a tubular portion of the implant device.

The tubular portion may have a generally flexible structure and may be configured to bend to facilitate navigation of the distal member through an aortic arch.

Some implementations of the present disclosure relate to an implant delivery shaft including: a distal portion configured to naturally assume a first width and expand beyond the first width in response to an implant device being inserted into the distal portion via an opening at a distal end of the distal portion; and a proximal portion configured to generally maintain the first width.

In some aspects, the techniques described herein relate to an implant delivery shaft, wherein the proximal portion is not configured to expand in response to the implant device extending into or against the proximal portion.

In some aspects, the techniques described herein relate to an implant delivery shaft or claim 23, wherein the distal portion includes one or more bendable rods configured to bend in response to the implant device being inserted into the distal portion.

In some aspects, the techniques described herein relate to an implant delivery shaft, wherein the distal portion further includes a first coating configured to extend across inner surfaces of the one or more bendable rods to form an inner diameter of the distal portion.

In some aspects, the techniques described herein relate to an implant delivery shaft, wherein the distal portion further includes a second coating configured to extend across outer surfaces of the one or more bendable rods to form an outer diameter of the distal portion.

In some aspects, the techniques described herein relate to an implant delivery shaft, wherein the one or more bendable rods are evenly spaced around a circumference of the distal portion.

In some aspects, the techniques described herein relate to an implant delivery shaft, wherein the one or more bendable rods extend longitudinally along the distal portion.

In some aspects, the techniques described herein relate to an implant delivery shaft, wherein the proximal portion includes a braided network of fibers.

In some aspects, the techniques described herein relate to an implant delivery shaft, wherein the proximal portion is configured to receive a tubular portion of the implant device.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example representation of a human heart in accordance with one or more examples.

FIG. 2 illustrates an example delivery system comprising one or more delivery devices configured to carry and/or percutaneously deliver one or more medical implants in accordance with one or more examples

FIG. 3 illustrates an example delivery system comprising one or more delivery devices configured to carry and/or percutaneously deliver one or more medical implants in accordance with one or more examples.

FIG. 4 illustrates an example delivery system comprising one or more delivery devices configured to carry and/or percutaneously deliver one or more medical implants in accordance with one or more examples.

FIGS. 5A and 5B illustrate side and frontal views, respectively, of an interior of at least a portion of an example delivery device in accordance with one or more examples.

FIG. 6 (FIGS. 6-1, 6-2, and 6-3) provides a flowchart illustrating a process including one or more steps for delivering one or more medical implants to one or more target locations (e.g., a left ventricle) within a heart, in accordance with one or more examples.

FIG. 7 (FIGS. 7-1, 7-2, and 7-3) provides images corresponding to steps of the process of FIG. 6.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for case of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for delivery of one or more medical implant devices (e.g., mechanical circulation systems) through various anatomy, which can include delivery via the aorta into the left ventricle. In some implementations, the present disclosure relates to delivery systems comprising expandable distal capsules which may be associated with and/or configured to receive and/or carry one or more medical implant devices. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly. Certain examples are disclosed herein in the context of cardiac implant devices. However, although certain principles disclosed herein are particularly applicable to the anatomy of the heart, it should be understood that implant device delivery systems in accordance with the present disclosure may be implanted in, or configured for implantation in, any suitable or desirable anatomy.

Some medical implant devices (e.g., mechanical circulatory systems) can be difficult to advance through delivery devices (e.g., sheaths) due at least in part to friction build-up and/or a lack of column strength from the delivery devices for push-ability. In some cases, a coating (e.g., a thermoplastic polyurethane coating) on a medical implant device can be damaged while the medical implant device is advanced through a delivery device.

Some examples described herein relate to an at least partially expandable delivery device configured to receive and/or protect one or more medical implant devices, which can include mechanical circulatory systems. A medical implant device can comprise multiple components. For example, a medical implant device can comprise a distal member (e.g., a motor) and/or a proximal member (e.g., in-flow and/or out-flow tubing) extending from and/or coupled to the distal member. In some examples, the distal member may have an increased diameter and/or width with respect to the proximal member. The increased width and/or diameter of the distal member may at least partially impede blood flow through various vessels during delivery of the medical implant device to a target location.

In some examples, a delivery device (e.g., a catheter, shaft, and/or tube) may comprise an expandable and/or distal portion configured to receive and/or expand at least partially around the distal member of a medical implant device. The delivery device may further comprise a proximal portion configured to approximately maintain a pre-determined width and/or diameter. In some examples, the distal portion of the delivery device may comprise a distal end of the delivery device. When the delivery device arrives at a target location, the medical implant device may be released from within the distal portion of the delivery device, which can cause the distal portion to naturally compress to a width and/or diameter approximately equivalent to a width and/or diameter of the proximal portion. In this way, the delivery device can assume and/or maintain a low and/or minimal profile while situated within the body. Moreover, a medical implant device can advantageously be placed into the distal end of a delivery device to advantageously avoid advancing the medical implant device through a length of the delivery device, which can cause damage to the medical implant device.

The medical implant device may be retracted into the delivery device following removal of the medical implant device from the delivery device. For example, the medical implant device and the delivery device may be tethered together via a guidewire and/or similar device extending at least partially through the delivery device and the medical implant device. The medical implant device may be retracted (e.g., by pulling on a proximal member of the medical implant device) until the medical implant device contacts a distal end and/or distal portion of the delivery device. The distal portion may be configured to expand in response to external force applied by the medical implant device at the distal end of the delivery device to allow the medical implant device to re-enter the distal portion.

### Cardiac Physiology

The anatomy of the heart is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

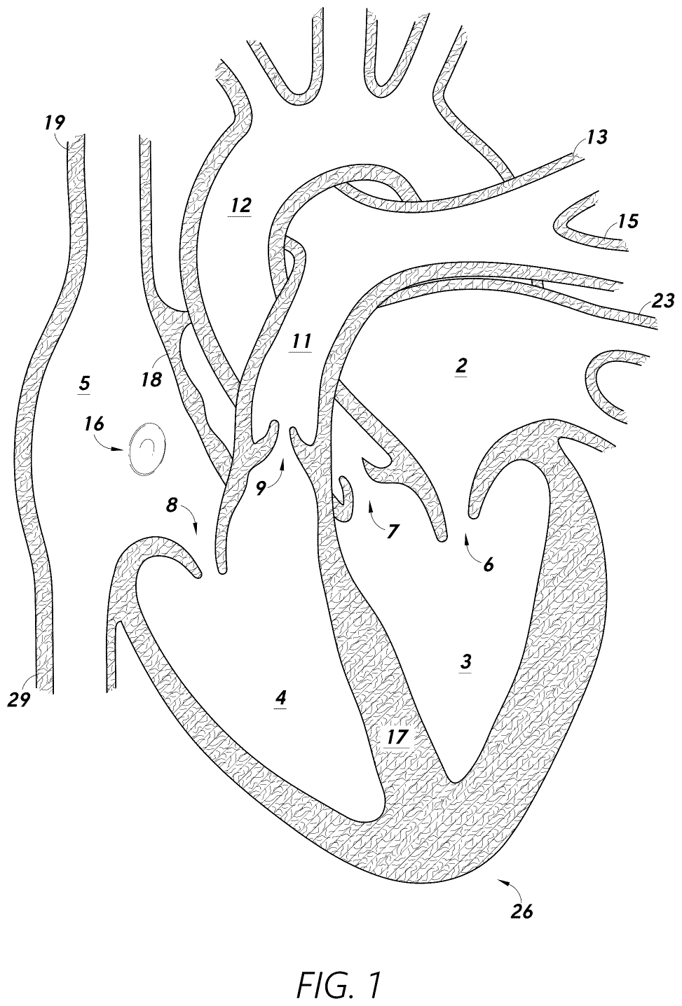

FIG. 1 illustrates an example representation of a heart 1 having various features relevant to certain examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left 15 and right 13 pulmonary arteries that branch off of the pulmonary trunk, as shown. The pulmonary veins 23 carry blood from the lungs to the left atrium 2.

In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps or leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Dysfunction of a heart valve and/or associated leaflets (e.g., pulmonary valve dysfunction) can result in valve leakage and/or other health complications.

The atrioventricular (i.e., mitral and tricuspid) heart valves may further comprise a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordac tendineac. A wall of muscle, referred to as the septum, separates the left-side chambers from the right-side chambers. In particular, an atrial septum wall portion 18 (referred to herein as the “atrial septum,” “interatrial septum,” or “septum”) separates the left atrium 2 from the right atrium 5, whereas a ventricular septum wall portion 17 (referred to herein as the “ventricular septum,” “interventricular septum,” or “septum”) separates the left ventricle 3 from the right ventricle 4. The inferior tip 26 of the heart 1 is referred to as the apex and is generally located on or near the midclavicular line, in the fifth intercostal space.

The coronary sinus 16 comprises a collection of veins joined together to form a large vessel that collects blood from the heart muscle (myocardium). The ostium of the coronary sinus, which can be guarded at least in part by a Thebesian valve in some patients, is open to the right atrium 5, as shown. The coronary sinus runs along a posterior aspect of the left atrium 2 and delivers less-oxygenated blood to the right atrium 5. The coronary sinus generally runs transversely in the left atrioventricular groove on the posterior side of the heart.

Any of several access pathways in the heart 1 may be utilized for maneuvering guidewires and catheters in and around the heart 1 to deploy implants and/or devices of the present application. For instance, access may be from above via either the subclavian vein or jugular vein into the superior vena cava (SVC) 19, right atrium 5, and from there into the coronary sinus 16. Alternatively, the access path may start in the femoral vein and through the inferior vena cava (IVC) 29 into the heart 1. Other access routes may also be used, and each can utilize a percutaneous incision through which the guidewire and catheter are inserted into the vasculature, normally through a sealed introducer, and from there the physician can control the distal ends of the devices from outside the body.

### Medical Implant Device Delivery Systems

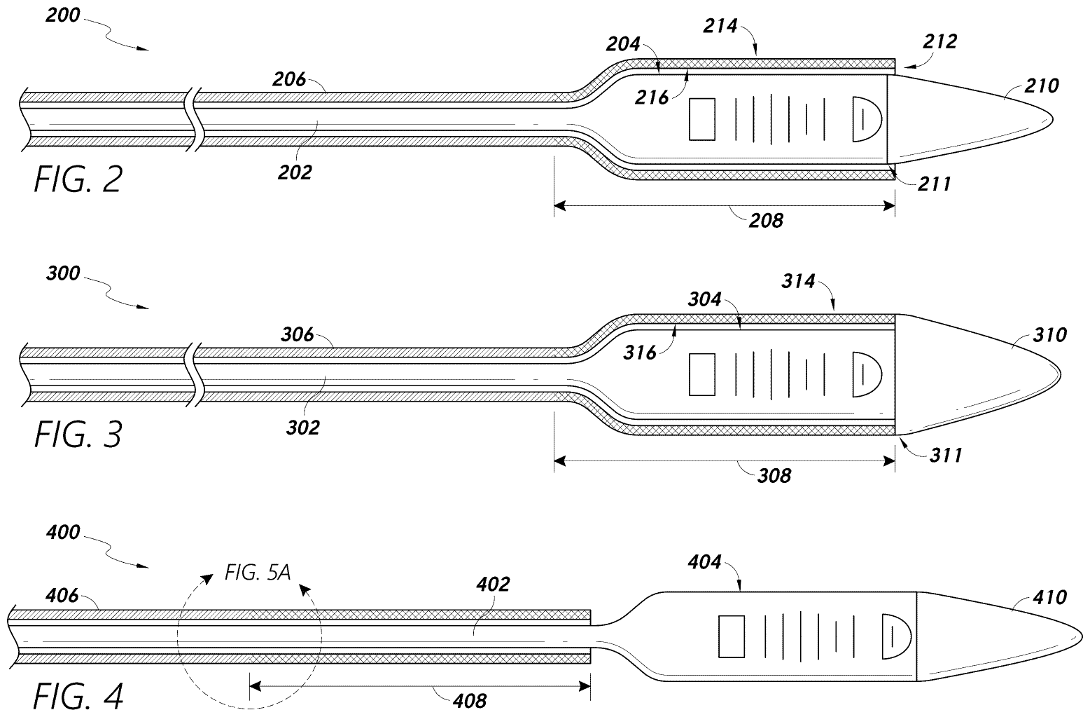

FIG. 2 illustrates an example delivery system 200 comprising one or more delivery devices configured to carry and/or percutaneously deliver one or more medical implants in accordance with one or more examples. A delivery device can comprise multiple components, which can include a fixed (e.g., proximal) portion 206 and/or an expandable (e.g., distal) portion 208. In some examples, the proximal portion 206 and the distal portion 208 can comprise different structures and/or materials.

In some examples, the proximal portion 206 may have a generally fixed width (e.g., a first width) and/or diameter and/or may be configured to generally maintain the fixed width and/or to resist deformation in response to external and/or internal forces from one or more implant devices and/or frictional forces during delivery through one or more anatomical areas. The proximal portion 206 may comprise an inner lumen having a generally fixed width and/or diameter. The inner lumen of the proximal portion 206 may be configured to receive at least portions of one or more implant devices. The proximal portion 206 and/or inner lumen of the proximal portion 206 may not be configured to expand in response to pressure from one or more implant devices having widths and/or diameters greater than the width and/or diameter of the proximal portion 206.

The distal portion 208 may have a generally flexible structure and/or may be configured to expand from a default width (e.g., the first width) and/or diameter (e.g., approximately equivalent to a width and/or diameter of the proximal portion 206) in response to forces applied by one or more implant devices. For example, one or more implant devices may be configured to pass through an inner lumen of the distal portion 208, as shown in FIG. 2. If a width and/or diameter of an implant device exceeds the default width and/or diameter of the distal portion 208, the distal portion 208 may be configured to naturally expand beyond the first width in response to pressure and/or force from the implant device. Expansion of the distal portion 208 may be driven by the implant device. For example, the distal portion 208 may be configured to expand only until a width and/or diameter is reached to allow the implant device to fit within the distal portion 208. The distal portion 208 may be configured to snugly fit around the implant device to minimize a profile of the delivery device while accommodating the implant device.

In some examples, the delivery device may be configured to carry and or deliver an implant device comprising multiple components, including a proximal member 202 (e.g., in-flow and/or out-flow tubing), a distal member 204, and/or a nose cone 210. The proximal member 202 may be coupled to the distal member 204 and/or may extend into the distal member 204. The distal member 204 may be coupled to the nose cone 210 and/or may extend into the nose cone 210.

The distal member 204 may have a greater width and/or diameter than the proximal member 202. For example, the distal member 204 may comprise a motor and/or other elements requiring an increased profile. The proximal member 202 may have a reduced width and/or diameter with respect to the distal member 204 to minimize a profile along a length of the implant device.

In some examples, the distal member 204 of the implant device may be configured to fit within the distal portion 208 of the delivery device and/or the distal portion 208 of the delivery device may be configured to hold the distal member 204 of the implant device. The proximal member 202 of the implant device may be configured to fit within the proximal portion 206 of the delivery device and/or the proximal portion 206 of the delivery device may be configured to hold the proximal member 202 of the implant device.

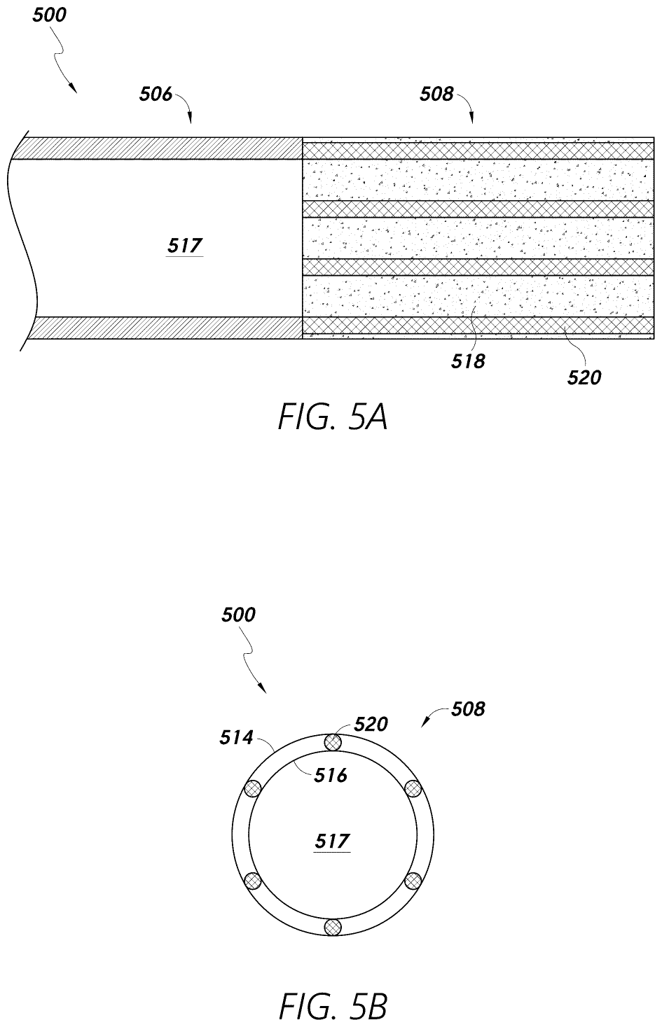

The width and/or diameter of the proximal portion 206 may be less than a width and/or diameter of the distal member 204 of the implant device. Moreover, the proximal portion 206 may have a generally rigid structure and/or may be configured to resist and/or prevent the distal member 204 from entering an inner lumen of the proximal portion 206. For example, as the distal member 204 is pulled into the distal portion 208 at a distal end 212 of the delivery device, the proximal member 202 may pass through the proximal portion 206 and/or the distal member 204 may press against a junction portion (see FIG. 5A) between the proximal portion 206 and the distal portion 208. The distal portion 208 may be configured to gradually reduce in diameter and/or width at the junction portion until the diameter and/or width is approximately equivalent to a diameter and/or width of the proximal portion 206.

In some examples, the distal end 212 of the delivery device and/or distal portion 208 may comprise a generally blunt surface configured to press against tissue, if necessary, without damaging the tissue. In the example shown in FIG. 2, the nose cone 210 may be configured to fit at least partially into the distal end 212 of the distal portion 208. For example, the distal member 204 and/or nose cone 210 may have a maximum diameter and/or width that may be less than an inner diameter 216 of the distal portion 208. Moreover, the maximum width and/or diameter of the nose cone 210 may be approximately equivalent to the diameter and/or width of the distal member 204 such that an edge 211 of the nose cone 210 may not extend beyond the distal member 204. In such examples, the distal end 212 of the delivery device and/or distal portion 208 may be configured to contact and/or dilate tissue as the delivery device and/or implant are delivered through a patient's body. The distal portion 208 and/or proximal portion 206 may have any suitable thickness between an outer diameter 214 and the inner diameter 216.

The nose cone 210 may be configured to fit at least partially within the distal portion 208. In this way, the nose cone 210 and/or the distal end 212 of the distal portion 208 may be configured to receive force applied to the delivery device and/or nose cone 210 as the delivery device and/or nose cone 210 are extended through the body and/or may be configured to shield the distal member 204 from such forces. The nose cone 210 may be configured to extend into contact with the inner diameter 216 and/or inner wall of the distal portion 208.

Although certain examples of delivery devices and/or implants disclosed herein comprise substantially circular cross-sections, in some examples, delivery devices and/or implants in accordance with the present disclosure may have oval-shaped, rectangular, diamond-shaped, or elliptical configurations.

In some examples, at least a portion of the implant may have a relatively delicate and/or fragile structure. For example, the distal member 204 may comprise one or more motors, wires, control circuitry, and/or various electronic components which may be susceptible to damage. Accordingly, at least a portion of the delivery device may be configured to protect the implant from damage during a delivery process. For example, the distal portion 208 may comprise a soft, durable, and/or reinforced structure configured to prevent damage to the implant.

The distal portion 208 may be configured to entirely and/or almost entirely encapsulate and/or surround the distal member 204 of the implant. For example, the distal member 204 of the implant may not be exposed to tissue as the delivery device delivers the implant to a target location within the body.

In some examples, the proximal portion 206 may be configured to translate pushing and/or pulling force at the proximal portion 206 to the distal portion 208. For example, extension of the proximal portion 206 may be configured to cause corresponding extension of the distal portion 208. Accordingly, the proximal portion 206 may comprise a generally rigid structure. In some examples, the proximal portion 206 may comprise a braided reinforced shaft and/or may be configured to maximize push force and/or column strength of the delivery device. For example, the proximal portion 206 can comprise one or more networks of braided and/or interwoven fibers. In some examples, the proximal portion 206 may have a generally rigid structure and/or may be resistant to increasing and/or decreasing in width and/or may be configured to generally maintain a default width even when implant devices are inserted through a lumen of the proximal portion 206, distal portion 208, and/or other portion of the delivery system 200.

The nose cone 210 may be affixed and/or coupled to the distal member 204 of the implant device and/or may be configured to provide a leading edge and/or end for the implant device and/or delivery device. In some examples, the nose cone 210 may comprise one or more suitable materials, including polyetheretherketone (PEEK), hydrometer, and/or polybutylene adipate terephthalate (PBAT).

In some examples, the proximal portion 206 and the distal portion 208 may be at least partially fused and/or coupled together. For example, the distal portion 208 may comprise one or more rods and/or coverings which may be configured to be fused to the proximal portion 206. The proximal portion 206 may comprise a reinforced and/or braided network of cords and/or fibers.

In some examples, the proximal member 202 may be configured to provide a lumen and/or container for various wires and/or cabling extending from the distal member 204. For example, the distal member 204 may comprise one or more motors and/or electronics having wiring configured to extend through the proximal member 202 and/or which may connect to one or more processing devices outside the body. The proximal member 202 may have a generally flimsy and/or flexible structure and/or the proximal portion 206 may be configured to receive and/or support the proximal member 202. In some examples, the proximal member 202 may have a generally tubular structure and/or may comprise a generally tubular portion of the implant device.

In some examples, an additional sheath (not shown) may be configured to extend at least partially over the delivery device and/or implant device during delivery into and/or through the body. For example, a sheath may extend over the delivery device and/or implant device during delivery through a major artery and/or vein. During delivery the delivery device and/or implant device may be extended at least partially beyond the sheath and/or may continue into the descending aorta and/or other anatomy. The implant device may then be configured to extend out of the delivery device in the descending aorta and/or similar anatomy.

FIG. 3 illustrates an example delivery system 300 comprising one or more delivery devices configured to carry and/or percutaneously deliver one or more medical implants in accordance with one or more examples. A delivery device can comprise multiple components, which can include a fixed (e.g., proximal) portion 306 and/or an expandable (e.g., distal) portion 308. In some examples, the proximal portion 306 and the distal portion 308 can comprise different structures and/or materials.

In some examples, the proximal portion 306 may have a generally fixed width and/or diameter and/or may be configured to resist deformation in response to external forces from one or more implant devices and/or frictional forces during delivery through one or more anatomical areas. The proximal portion 306 may comprise an inner lumen having a generally fixed width and/or diameter. The inner lumen of the proximal portion 306 may be configured to receive at least portions of one or more implant devices. The proximal portion 306 and/or inner lumen of the proximal portion 306 may not be configured to expand in response to pressure from one or more implant devices having widths and/or diameters greater than the width and/or diameter of the proximal portion 306.

The distal portion 308 may have a generally flexible structure and/or may be configured to expand from a default width and/or diameter (e.g., approximately equivalent to a width and/or diameter of the proximal portion 306) in response to forces applied by one or more implant devices. For example, one or more implant devices may be configured to pass through an inner lumen of the distal portion 308, as shown in FIG. 3. If a width and/or diameter of an implant device exceeds the default width and/or diameter of the distal portion 308, the distal portion 308 may be configured to naturally expand in response to pressure and/or force from the implant device. Expansion of the distal portion 308 may be driven by the implant device. For example, the distal portion 308 may be configured to expand only until a width and/or diameter is reached to allow the implant device to fit within the distal portion 308. The distal portion 308 may be configured to snugly fit around the implant device to minimize a profile of the delivery device while accommodating the implant device.

In some examples, the delivery device may be configured to carry and or deliver an implant device comprising multiple components, including a proximal member 302 (e.g., in-flow and/or out-flow tubing), a distal member 304, and/or a nose cone 310. The proximal member 302 may be coupled to the distal member 304 and/or may extend into the distal member 304. The distal member 304 may be coupled to the nose cone 310 and/or may extend into the nose cone 310.

The distal member 304 may have a greater width and/or diameter than the proximal member 302. For example, the distal member 304 may comprise a motor and/or other elements requiring an increased profile. The proximal member 302 may have a reduced width and/or diameter with respect to the distal member 304 to minimize a profile along a length of the implant device.

In some examples, the distal member 304 of the implant device may be configured to fit within the distal portion 308 of the delivery device and/or the distal portion 308 of the delivery device may be configured to hold the distal member 304 of the implant device. The proximal member 302 of the implant device may be configured to fit within the proximal portion 306 of the delivery device and/or the proximal portion 306 of the delivery device may be configured to hold the proximal member 302 of the implant device.

The width and/or diameter of the proximal portion 306 may be less than a width and/or diameter of the distal member 304 of the implant device. Moreover, the proximal portion 306 may have a generally rigid structure and/or may be configured to resist and/or prevent the distal member 304 from entering an inner lumen of the proximal portion 306. For example, as the distal member 304 is pulled into the distal portion 308 at a distal end of the delivery device, the proximal member 302 may pass through the proximal portion 306 and/or the distal member 304 may press against a junction portion (see FIG. 5A) between the proximal portion 306 and the distal portion 308. The distal portion 308 may be configured to gradually reduce in diameter and/or width at the junction portion until the diameter and/or width is approximately equivalent to a diameter and/or width of the proximal portion 306.

In some examples, the nose cone 310 may be configured to extend at least partially over the distal end of the distal portion 308. For example, the nose cone 310 may have a maximum diameter and/or width that may be greater than an inner diameter 316 of the distal portion 308. Moreover, the maximum width and/or diameter of the nose cone 310 may be greater than the diameter and/or width of the distal member 304 such that an edge 311 of the nose cone 310 may extend at least partially beyond the distal member 304. In such examples, the nose cone 310 may be configured to prevent the distal end of the delivery device and/or distal portion 308 from contacting tissue as the delivery device and/or implant are delivered through a patient's body. The distal portion 308 and/or proximal portion 306 may have any suitable thickness between an outer diameter 314 and the inner diameter 316. In some examples, a maximum diameter and/or width of the nose cone 310 may be approximately equivalent to the outer diameter 314 when the distal member 304 is situated within the distal portion 308.

The edge 311 of the nose cone 310 may be configured to form a step configured to extend at least partially over one or more edges of the distal portion 308. As a result, force applied to the nose cone 310 by tissue and/or blood flow may be translated through the nose cone 310 to the distal portion 308 and/or may not be translated to the distal member 304 of the implant. Moreover, the distal portion 308 may be configured to support the nose cone 310 at the edge 311. The nose cone 310 may be configured to not significantly compress, thereby minimizing and/or avoiding translation of force to the distal member 304.

FIG. 4 illustrates an example delivery system 400 comprising one or more delivery devices configured to carry and/or percutaneously deliver one or more medical implants in accordance with one or more examples. A delivery device can comprise multiple components, which can include a fixed (e.g., proximal) portion 406 and/or an expandable (e.g., distal) portion 408. In some examples, the proximal portion 406 and the distal portion 408 can comprise different structures and/or materials.

As shown in FIG. 4, at least a portion of the implant (e.g., the distal member 404 and/or nose cone 410) may be configured to exit the delivery device from a distal end of the delivery device. When the distal member 404 is not situated within the distal portion 408, the distal portion 408 may be configured to naturally compress and/or reduce to a width and/or diameter approximately equivalent to a width and/or diameter of the proximal portion 406. In this way, the delivery device may be configured to present a minimal profile while situated within a patient's body.

In some examples, the proximal portion 406 may have a generally fixed width and/or diameter and/or may be configured to resist deformation in response to external forces from one or more implant devices and/or frictional forces during delivery through one or more anatomical areas. The proximal portion 406 may comprise an inner lumen having a generally fixed width and/or diameter. The inner lumen of the proximal portion 406 may be configured to receive at least portions of one or more implant devices. The proximal portion 406 and/or inner lumen of the proximal portion 406 may not be configured to expand in response to pressure from one or more implant devices having widths and/or diameters greater than the width and/or diameter of the proximal portion 406.

The distal portion 408 may have a generally flexible structure and/or may be configured to expand from a default width and/or diameter (e.g., approximately equivalent to a width and/or diameter of the proximal portion 406) in response to forces applied by one or more implant devices. For example, one or more implant devices may be configured to pass through an inner lumen of the distal portion 408, as shown in FIG. 4. If a width and/or diameter of an implant device exceeds the default width and/or diameter of the distal portion 408, the distal portion 408 may be configured to naturally expand in response to pressure and/or force from the implant device. Expansion of the distal portion 408 may be driven by the implant device. For example, the distal portion 408 may be configured to expand only until a width and/or diameter is reached to allow the implant device to fit within the distal portion 408. The distal portion 408 may be configured to snugly fit around the implant device to minimize a profile of the delivery device while accommodating the implant device.

In some examples, the delivery device may be configured to carry and or deliver an implant device comprising multiple components, including a proximal member 402 (e.g., in-flow and/or out-flow tubing), a distal member 404, and/or a nose cone 410. The proximal member 402 may be coupled to the distal member 404 and/or may extend into the distal member 404. The distal member 404 may be coupled to the nose cone 410 and/or may extend into the nose cone 410.

The distal member 404 may have a greater width and/or diameter than the proximal member 402. For example, the distal member 404 may comprise a motor and/or other elements requiring an increased profile. The proximal member 402 may have a reduced width and/or diameter with respect to the distal member 404 to minimize a profile along a length of the implant device.

In some examples, the distal member 404 of the implant device may be configured to fit within the distal portion 408 of the delivery device and/or the distal portion 408 of the delivery device may be configured to hold the distal member 404 of the implant device. The proximal member 402 of the implant device may be configured to fit within the proximal portion 406 of the delivery device and/or the proximal portion 406 of the delivery device may be configured to hold the proximal member 402 of the implant device.

The width and/or diameter of the proximal portion 406 may be less than a width and/or diameter of the distal member 404 of the implant device. Moreover, the proximal portion 406 may have a generally rigid structure and/or may be configured to resist and/or prevent the distal member 404 from entering an inner lumen of the proximal portion 406. For example, as the distal member 404 is pulled into the distal portion 408 at a distal end of the delivery device, the proximal member 402 may pass through the proximal portion 406 and/or the distal member 404 may press against a junction portion (see FIG. 5A) between the proximal portion 406 and the distal portion 408. The distal portion 408 may be configured to gradually reduce in diameter and/or width at the junction portion until the diameter and/or width is approximately equivalent to a diameter and/or width of the proximal portion 406.

In some examples, the distal end of the delivery device and/or distal portion 408 may comprise a generally blunt surface configured to press against tissue, if necessary, without damaging the tissue. The distal portion 408 and/or proximal portion 406 may have any suitable thickness between an outer diameter and the inner diameter.

The distal portion 408 may be configured to naturally and/or elastically collapse to the compressed configuration shown in FIG. 4 following removal of the distal member 204 of the implant from the distal portion 408 and/or the distal portion 408 may be configured to be manually compressed. Compression of the distal portion 408 may allow the delivery device to prevent blood obstruction through one or more blood vessels.

FIGS. 5A and 5B illustrate side and frontal views, respectively, of an interior of at least a portion of an example delivery device 500 in accordance with one or more examples. In some examples, the delivery device 500 may be at least partially composed of one or more bendable, malleable, and/or flexible rods 520 extending at least partially along a length of the delivery device 500. For example, a distal portion 508 of the delivery device 500 may comprise one or more (e.g., six) rods 520. In some examples, a proximal portion 506 of the delivery device 500 may also comprise one or more rods 520 or may not comprise any rods 520. In some examples, the one or more rods 520 may be at least partially enclosed by one or more coverings 518 configured to close gaps between individual rods 520. For illustrative purposes, FIGS. 5A and 5B provide internal views in which at least a portion of the coverings 518 and/or other components of the delivery device 500 may be see-through.

The delivery device 500 may comprise an inner lumen 517 extending through the proximal portion 506 and/or distal portion 508. In some examples, the delivery device 500 may have a generally tubular and/or cylindrical body and/or the inner lumen 517 may have a cylindrical and/or tubular shape. The one or more rods 520 may be configured to extend longitudinally along the delivery device 500, distal portion 508, and/or lumen 517.

While the delivery device 500 is illustrated comprising six rods 520, the delivery device 500 can comprise any number of rods 520. The rods 520 may be situated approximately equidistant from each other along a circumferential surface of the delivery device 500 and/or distal portion 508. In some examples, the delivery device 500 and/or distal portion 508 may comprise an inner diameter 516 and/or outer diameter 514 composed of one or more coverings 518 configured to cover the one or more rods 520 and/or to close gaps between the one or more rods 520. In some examples, the rods 520 and/or coverings 518 may have a generally soft structure to provide cushioning to the distal member of the implant device and/or to otherwise prevent unnecessary force on the distal member of the implant device.

The rods 520 may comprise any suitable materials, which can include nylon and/or Nitinol. In some examples, the rods 520 may be configured to bend and/or elastically return to a shape-set form (e.g., a generally flat and/or linear form). The rods 520 may have any suitable shape and/or may have a generally circular cross-section. The covering(s) 518 may be composed of any suitable materials, which can include NeoSoft and/or similar materials. The covering(s) 518 may be configured to stretch and/or expand as the rods 520 are bent and/or moved. In some examples, a NeoSoft and/or similar coating 518 may be applied over the rods 520 to form an inner diameter 516 of the distal portion 508 and/or additional hydrophilic coatings may be applied over the rods 520 to form an outer diameter 514 of the distal portion 508. The delivery device 500 may comprise a space between the inner diameter 516 and the outer diameter 514 and/or one or more covering materials may fill the space between the inner diameter 516 and the outer diameter 514. The covering(s) 518 and/or coatings may form a generally tubular shaft and/or one or more rods 520 may be configured to extend between the inner and/or outer surfaces of the tubular shaft.

The bendable rods 520 may be configured to bend in response to an implant device being inserted at least partially into the distal portion 508 of the delivery device. For example, the implant device may have a greater width than a default and/or resting width of the proximal portion 506 and/or distal portion 508. Accordingly, the rods 520 may be configured to bend to increase a width of the distal portion 508 such that the distal portion 508 can accommodate at least a portion of the implant device.

In some examples, the rods 520 may be configured to provide column support to the delivery device and/or to translate pushing and/or pulling force from the proximal portion 206. The rods 520 may form a cage configured to at least partially contain the distal member of a medical implant and/or the one or more coverings 518 may be configured to coat and/or extend between the rods 520. In some examples, the one or more rods 520 may be spaced approximately evenly spaced around a circumference of the distal portion 508, as shown in FIG. 5B. As the rods 520 bend to allow expansion of the distal portion 508, distances between the rods 520 may increase.

The proximal portion 506 may be configured to be fused and/or coupled to the distal portion 508. For example, the proximal portion 506 may be configured to fused to one or more rods 520 and/or coverings 518 of the distal portion 508. In some examples, a heat lamination and/or overmolding process may be sued to couple the proximal portion 506 to the distal portion 508.

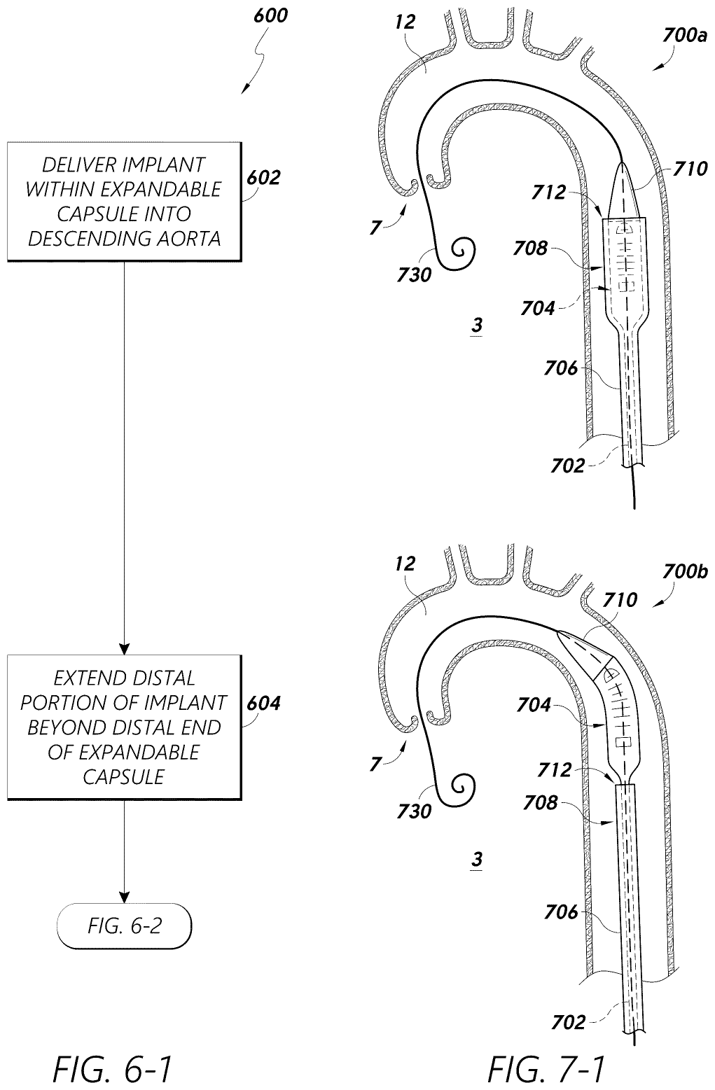

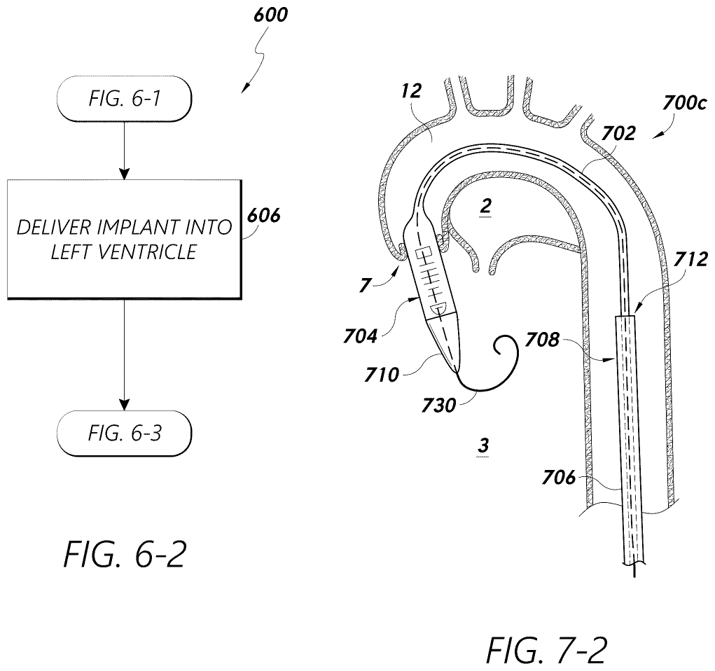

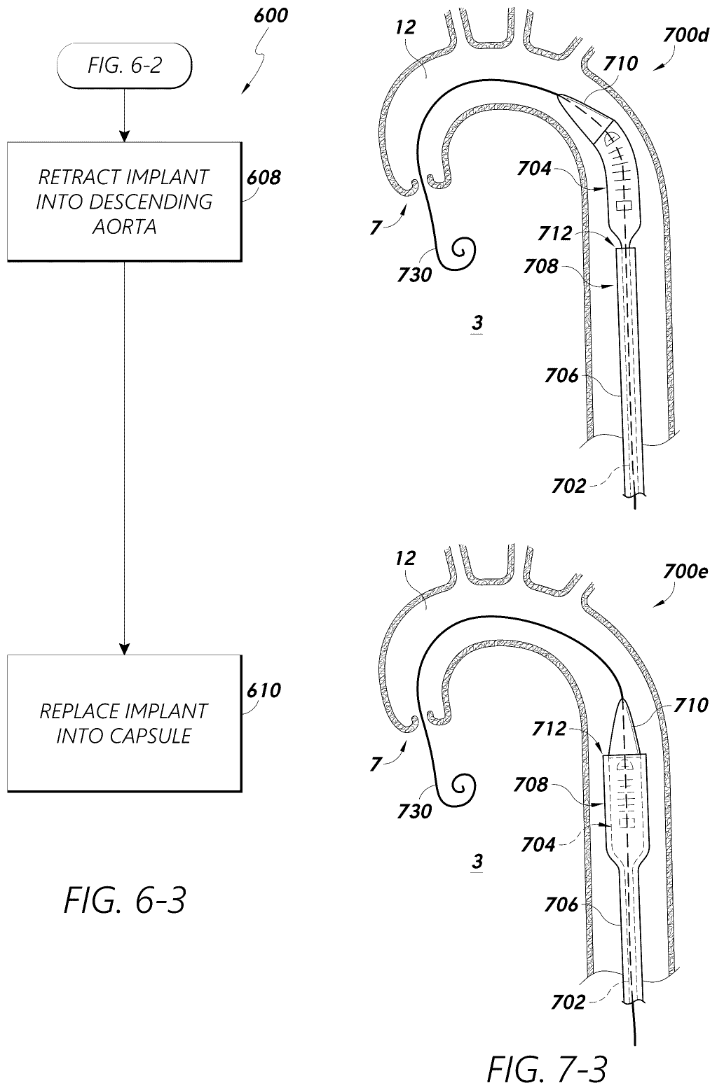

FIG. 6 (FIGS. 6-1, 6-2, and 6-3) provides a flowchart illustrating a process 600 including one or more steps for delivering one or more medical implants to one or more target locations (e.g., a left ventricle 3) within a heart, in accordance with one or more examples. FIG. 7 (FIGS. 7-1, 7-2, and 7-3) provides images corresponding to steps of the process 600 of FIG. 6.

At step 602, the process 600 involves delivering a medical implant at least partially within a distal portion 708 (e.g., capsule) of a delivery device into a descending aorta of an aorta 12, as shown in image 700a of FIG. 7. The delivery device may comprise a distal portion 708 and/or a proximal portion 706. The distal portion 708 comprise a distal end 712 and/or may be situated nearer the distal end 712 than the proximal portion 706.

In some examples, the medical implant may comprise multiple components. For example, the medical implant can comprise a proximal member 702, a distal member 704, and/or a nose cone 710. The proximal member 702 may comprise a generally thin tubular portion, which may be configured to convey circuitry and/or fluids to and/or from the distal member 704. The distal member 704 can have an increased width and/or diameter with respect to the proximal member 702 and/or may be situated at least partially between the proximal member 702 and the nose cone 710.

The distal member 704 may be situated at least partially within the distal portion 708 of the delivery device and/or the proximal member 702 may be situated at least partially within the proximal portion 706 of the delivery device during delivery. The nose cone 710 may extend at least partially beyond the distal end 712 of the delivery device during delivery.

In some examples, the delivery device and/or medical implant may be configured to track along a guidewire 730. For example, the guidewire 730 may be delivered via the aorta 12 and/or may gain access to the left ventricle 3 during an earlier delivery process. The delivery device and/or implant may then follow the guidewire 730. In some examples, the guidewire 730 may be configured to pass through lumens of the nose cone 710, distal member 704, proximal member 702, distal portion 708, and/or proximal portion 706.

The distal portion 708 may be configured to assume a relatively expanded profile while the distal member 704 of the implant is situated at least partially within the distal portion 708. For example, the distal portion 708 may be configured to stretch, expand, and/or bend in response to force applied by the distal member 704 at an inner diameter of the distal portion 708. The distal portion 708 may comprise one or more flexible rods and/or coverings configured to elastically deform in response to pressure from the distal member 704.

At step 604, the process 600 involves extending the implant at least partially beyond the distal end 712 of the delivery device and/or distal portion 708, as shown in image 700b of FIG. 7. For example, the proximal member 702 may be configured to be extended to cause corresponding extension of the distal member 704 beyond the distal end 712. As the distal member 704 exits the delivery device, the distal portion 708 may elastically return to a default and/or compressed form, in which a width and/or diameter of the distal portion 708 may be less than a width and/or diameter of the distal member 704 and/or in which the width and/or diameter of the distal portion 708 may be approximately equal to a width and/or diameter of the proximal portion 706.

At step 606, the process 600 involves delivering the implant along the aortic arch of the aorta 12 and/or through the aortic valve 7 into the left ventricle 3, as shown in image 700c of FIG. 7. The delivery device may be configured to remain in a relatively compressed form within the descending aorta while the implant device is delivered into the left ventricle 3.

As shown in image 700c, the proximal member 702 of the delivery device may be configured to bend to facilitate navigation of the distal member 704 through patient anatomy and/or to a target location. For example, the proximal member 702 may be configured to bend around an aortic arch of the aorta 12 to allow the distal member 704 to traverse the aorta 12 and/or to reach the aortic valve 7. In some examples, the proximal member 702 may have a generally tubular and/or flexible structure.

At step 608, the process 600 involves retracting the implant back through the aortic valve 7 and/or into the descending aorta of the aorta 12, as shown in image 700d of FIG. 7.

At step 610, the process 600 involves retracting and/or replacing the distal member 704 of the implant into the distal portion 708 of the delivery device, as shown in image 700e of FIG. 7. The distal portion 708 may be configured to expand and/or increase in width and/or diameter in response to the distal member 704 entering the distal portion 708. The proximal portion 706 of the delivery device may not be configured to expand in response to the distal member 704 retracting into the distal portion 708 and/or the proximal portion 706 may be configured to maintain a fixed width and/or diameter as the distal member 704 enters the distal portion 708. In some examples, the distal member 704 may be retracted until the distal member 704 presses against the distal end 712 of the delivery device, which may cause the distal end 712 and/or distal portion 708 to expand.

### ADDITIONAL EXAMPLES

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An implant delivery system comprising:
a distal portion configured to naturally assume a first width and expand beyond the first width in response to an implant device being inserted into the distal portion via an opening at a distal end of the distal portion; and
a proximal portion configured to generally maintain the first width.

2. The implant delivery system of claim 1, wherein the proximal portion is not configured to expand in response to the implant device extending into or against the proximal portion.

3. The implant delivery system of claim 1, wherein the distal portion comprises one or more bendable rods configured to bend in response to the implant device being inserted into the distal portion.

4. The implant delivery system of claim 3, wherein the distal portion further comprises a first coating configured to extend across inner surfaces of the one or more bendable rods to form an inner diameter of the distal portion.

5. The implant delivery system of claim 4, wherein the distal portion further comprises a second coating configured to extend across outer surfaces of the one or more bendable rods to form an outer diameter of the distal portion.

6. The implant delivery system of claim 3, wherein the one or more bendable rods extend longitudinally along the distal portion.

7. The implant delivery system of claim 1, wherein the proximal portion comprises a braided network of fibers.

8. The implant delivery system of claim 1, further comprising a nose cone configured to provide a leading end for the implant device.

9. The implant delivery system of claim 8, wherein the nose cone is configured to extend at least partially over the distal end of the distal portion.

10. The implant delivery system of claim 1, wherein the proximal portion is configured to receive a tubular portion of the implant device.

11. A method comprising:
delivering a delivery device carrying an implant device into a blood vessel, the delivery device comprising:
a distal portion configured to naturally assume a first width and expand beyond the first width in response to a distal member of the implant device being inserted into the distal portion via an opening at a distal end of the distal portion; and
a proximal portion configured to generally maintain the first width;
extending at least the distal member of the implant device beyond the distal end of the delivery device to cause the distal portion of the delivery device to assume the first width; and
retracting at least the distal member of the implant device into the distal portion of the delivery device to cause the distal portion of the delivery device to expand beyond the first width.

12. The method of claim 11, wherein the proximal portion is not configured to expand in response to the distal member of the implant device retracting into the distal portion of the delivery device.

13. The method of claim 11, wherein the distal portion comprises one or more bendable rods configured to bend in response to the implant device being inserted into the distal portion.

14. The method of claim 11, wherein the proximal portion comprises a braided network of fibers.

15. The method of claim 11, wherein the proximal portion is configured to receive a tubular portion of the implant device.

16. An implant delivery shaft comprising:
a distal portion configured to naturally assume a first width and expand beyond the first width in response to an implant device being inserted into the distal portion via an opening at a distal end of the distal portion; and
a proximal portion configured to generally maintain the first width.

17. The implant delivery shaft of claim 16, wherein the proximal portion is not configured to expand in response to the implant device extending into or against the proximal portion.

18. The implant delivery shaft of claim 16, wherein the distal portion comprises one or more bendable rods configured to bend in response to the implant device being inserted into the distal portion.

19. The implant delivery shaft of claim 16, wherein the proximal portion comprises a braided network of fibers.

20. The implant delivery shaft of claim 16, wherein the proximal portion is configured to receive a tubular portion of the implant device.
